(19) United States
(12) Patent Application Publication
(10) Pub. No.: US 2017/0002168 A1
(43) Pub. Date: Jan. 5, 2017

(54) LOW TEMPERATURE PROCESSES FOR PRODUCING DRY CELLULOSE BASED FOAMS

(71) Applicants: DOW GLOBAL TECHNOLOGIES LLC, Midland, MI (US); ROHM AND HAAS COMPANY, Philadelphia, PA (US)

(72) Inventors: Jaime L. CURTIS-FISK, Midland, MI (US); Paul J. SHESKEY, Midland, MI (US); Michael BUCHMAN, Midland, MI (US); Andrea C. KEENAN, Collegeville, PA (US); Kinjalbahen JOSHI, Collegeville, PA (US); Jodi A. THOMAS, Midland, MI (US)

(21) Appl. No.: 15/100,029

(22) PCT Filed: Nov. 20, 2014
(86) PCT No.: PCT/US2014/066655

§ 371 Date: May 27, 2016

Related U.S. Application Data
(60) Provisional application No. 61/909,889, filed on Nov. 27, 2013.

(51) Int. Cl. C08J 9/28; A61K 47/38; A61Q 5/02; C08J 9/00; A61K 8/86; A61K 8/73; A61K 31/375; A61K 47/10

(52) U.S. Cl. CPC C08J 9/28; A61K 31/375; A61K 47/38; A61K 47/10; C08J 9/0061; A61K 8/86; A61K 8/732; A61K 8/731; A61Q 5/02; C08J 2301/28; C08J 2471/02; C08J 2207/10; C08J 2205/10; C08J 2201/05

(57) ABSTRACT

A composition comprising a gelling agent is provided. The gelling agent consists essentially of a cellulose ether based gelling polymer and the composition is a rigid foam at 20 degrees Celsius. The composition may also comprise an active ingredient. Also provided is a process of making the composition comprising drying a foamed liquid composition comprising the gelling agent at low temperatures. The foamed liquid composition may also comprise an active ingredient.

### CROSS-REFERENCE TO RELATED APPLICATION

This application claims priority to U.S. Provisional Application No. 61/909,889, filed Nov. 27, 2013, the disclosure of which is hereby incorporated by reference in its entirety.

### FIELD

This disclosure relates to foam compositions and processes for making such compositions.

### BACKGROUND

In the personal care industry, active ingredients may be delivered in skin treatments by formulating the active into a cream or lotion, then applying to the skin. It is sometimes difficult to either load enough active ingredient into a stable formulation to be effective or to deliver the active ingredient from the formulation onto the skin. The active ingredient is therefore not available on the skin for efficacy. For example, in compositions where the active ingredient is in a skin care product such as a lotion, it might not be possible to apply enough lotion at the treatment site to get a benefit.

In addition, active ingredients used in the personal care industry are often temperature sensitive. Botanical products in particular can be difficult to formulate, as most are heat sensitive and have low solubility and stability. It is also desirable to add incompatible active ingredients to formulations, for example, hydrophobic and hydrophilic components. Furthermore, while active ingredients can be incorporated into water-soluble films, these films need to be manipulated in water such that they dissolve before they are applied. Thus, the active ingredient can become diluted if too much water is used, or the film may not fully dissolve to release the active ingredient if not enough water is used.

Therefore, there is a need for a stable formulation that is able to incorporate heat sensitive active ingredients and that can ensure a targeted delivery of the active ingredient to the desired location. Such a formulation may be useful in many industries where targeted delivery is desired, including but not limited to personal care and laundry/stain removal applications.

### BRIEF SUMMARY

In one aspect, a composition comprising a gelling agent is provided. The gelling agent consists essentially of a cellulose based gelling polymer, wherein the cellulose based gelling polymer has a viscosity of between about 2 and about 4000 centipoise in a 2 percent solution at about 20 degrees Celsius. The composition can be a rigid foam at about 20 degrees Celsius. The composition may also comprise an active ingredient

In another aspect, a process of making such a composition is provided. The process comprises drying a foamed liquid composition comprising the gelling agent at a temperature between about 40 and about 50 degrees Celsius for between about 10 and about 30 minutes. The foamed liquid composition may also comprise an active ingredient.

### DETAILED DESCRIPTION

It is beneficial to have an aerated foam that dissolves quickly to ensure proper placement and dose of the active ingredient. A light, stable formulation is desired for targeted delivery. It is also desired to maintain the active ingredient in a stable state without the need to develop a complex formulation to obtain a long shelf life.

Rapidly dissolving solid dosage forms for delivery of personal care active ingredients can be generated by drying a foamed liquid mixture of the active ingredient and a gelling agent, such as a cellulose based gelling polymer. The present disclosure provides a composition comprising a gelling agent. The composition may also comprise an active ingredient. The gelling agent may consist essentially of a cellulose based gelling polymer, wherein the cellulose based gelling polymer has a viscosity of between about 2 and about 4000 centipoise (cP), preferably between about 2 and about 100 cP, and more preferably between about 2 and about 10 cP in a 2 weight percent solution at about 20 degrees Celsius. For example, the cellulose based gelling polymer may be hydroxypropyl methylcellulose (HPMC), methylcellulose, or a combination thereof. In some embodiments, the cellulose based gelling polymer may have a viscosity of about 6 cP in a 2 weight percent solution at about 20 degrees Celsius, for example. In other embodiments, the cellulose based gelling polymer may have a viscosity of about 2.5 cP in a 2 weight percent solution at about 20 degrees Celsius. The cellulose based gelling polymer may be the only gelling agent used in the composition. For example, the composition may include only HPMC and no other gelling agent. In other embodiments, the composition may include only methylcellulose or HPMC and methylcellulose.

Incorporating a gas, for example, air, into the solution increases the porosity and surface area of the resulting solid, which is a key characteristic for liquid to quickly enter the solid and dissolve the material. The composition may comprise between about 20 and about 99 percent, and preferably between about 30 and about 70 percent, air by volume. The amount of air desired may be application specific. For example, if a more rigid foam is needed, it may be desirable to have less air. The amount of air may also be dependent on the composition of the active ingredient used. For example, when the active ingredient is a solid pigment, it may be necessary to include more air. In some embodiments, the composition may comprise, by volume, about 50 percent air and 50 percent of the composition.

The composition may be capable of dissolving or dispersing quickly in order to provide rapid delivery of the active ingredient. For example, the composition may be dispersible in water within about 10 seconds with agitation. In some embodiments, the composition may be used to provide an active ingredient to a target, such as skin. For example, such compositions may be used for leave-on skin care treatment including but not limited to pharmaceutical treatment, cosmetics, lotions, acne treatment, and vitamin C treatment. In other embodiments, such compositions may be used for rinse-off products including but not limited to shampoo, body wash, shaving cream, and facial cleansers. In further embodiments, the composition may be used to provide an active ingredient used in cleaning, such as in laundry applications and stain removal. The composition may also disintegrate (i.e., break into small pieces) without the use of water. For example, it may be desirable to add an oil-absorbing agent such as cornstarch as an active ingredient to the composition in order to absorb oil in hair.

The gelling agent may undergo rapid thermal gelation upon heating, allowing the foam to maintain its structure and hold suspended solids during the drying process. This produces a solid foam with greater mechanical integrity than freeze-drying of a solution and with greater porosity than film strips, two alternative approaches commonly used in, e.g., the pharmaceutical industry, to produce this type of product. The composition may be a rigid foam at room temperature (about 20 degrees Celsius). Thus, the composition may be suitable for individual packaging and storage for a variety of applications.

Also disclosed is a process of making the composition of the present invention. The process comprises drying a foamed liquid composition comprising the gelling agent at a temperature between about 40 and about 50 degrees Celsius. The foamed liquid composition may also comprise an active ingredient. The liquid composition may be dried, e.g., for between about 10 and about 30 minutes. In some embodiments, the drying time will be faster, for example, if smaller foam compositions are desired. In certain, the process does not require the presence of a surfactant, resulting in compositions free or substantially free of surfactant. In other embodiments, a surfactant may be added to the composition during the process. The surfactant may be used to assist in creating the foam or the surfactant may be added as an active ingredient.

Low temperature gelling water-soluble methylcellulose and HPMC polymers begin the gelation process at room temperature, and once gelled, the resulting foam holds structure during drying. This process can be conducted at much lower temperatures than required for extrusion, an alternative method of producing a dry solid foam composition. This may be of particular benefit for temperature sensitive active ingredients that may not survive high temperature processes such as extrusion. For example, the active ingredient may be susceptible to degradation at high temperatures. In some embodiments, this process may be amenable to active ingredients that show degradation above 50 degrees Celsius. The active ingredient may be, for example, a pigment, vitamin C, a moisturizer, an acne-treating agent, or a botanical product, such as aloe vera or tea tree oil. In other embodiments, the active ingredient may be a cleaning agent, such as cleaning agents used in personal care cleansers (for example, shampoo, body wash, facial cleansers) or in household cleaning (for example, laundry).

### EXAMPLES

Dry foams are prepared by foaming an aqueous solution or suspension of HPMC and, in some examples, an active ingredient. A foam quality of 50% (by volume, 50% air, 50% formulation) is used in the examples shown in Table 1, but foam quality is also demonstrated between 30% and 70% air by volume while still producing a high quality product, i.e., rigid foams with high porosity and the ability to quickly and accurately deliver active ingredients. The foam is dropped onto a non-stick baking sheet and dried at 50° C. for 20 minutes. The composition formulations are shown in Table 1 below. Such formulations result in rigid foams that are able to quickly and accurately deliver active ingredients to a target.

TABLE 1
Example Formulations

| Example No. | Formulation (weight percentage) |
|---|---|
| 1 | 48% HPMC composition (6 cP viscosity), 48% pigment |
| | (active ingredient), 4% PEG 400 (plasticizer) |
| 2 | 46% HPMC composition (6 cP viscosity), 46% pigment |
| | (active ingredient), 8% PEG 400 (plasticizer) |
| 3 | 33% HPMC composition (2.5 cP viscosity), 66% surfactant |
| | blend |
| 4 | 46% HPMC composition (2.5 cP viscosity), 46% |
| | cornstarch, 8% PEG 400 (plasticizer) |
| 5 | 82% HPMC composition (2.5 cP viscosity), 2% ascorbic |
| | acid (active ingredient), 16% PEG 400 (plasticizer) |

In examples 1, 2, 4, and 5, a plasticizer is used to prevent the composition from being brittle and to hold the structure of the foam. In example 3, a surfactant is used as a foaming agent for shampooing applications. However, the surfactant is not required in order to create the rigid formulation, meaning that the only gelling agent is the HPMC. In example 4, cornstarch is used as an oil-absorbing agent to create a “dry” shampoo, which disintegrates without water.

While the invention has been described above according to its preferred embodiments, it can be modified within the spirit and scope of this disclosure. This application is therefore intended to cover any variations, uses, or adaptations of the invention using the general principles disclosed herein. Further, the application is intended to cover such departures from the present disclosure as come within the known or customary practice in the art to which this invention pertains and which fall within the limits of the following claims.

1. A composition comprising:
a gelling agent, wherein the gelling agent consists essentially of a cellulose based gelling polymer,
wherein the cellulose based gelling polymer has a viscosity of between about 2 and about 4000 centipoise in a 2 percent by weight solution at about 20 degrees Celsius, and
wherein the composition is a rigid foam at about 20 degrees Celsius.

2. The composition of claim 1, further comprising an active ingredient.

3. The composition of claim 1 or claim 2, wherein the cellulose based gelling polymer is hydroxypropyl methylcellulose, methylcellulose, or a combination thereof.

4. The composition of claim 1, wherein the cellulose based gelling polymer has a viscosity of between about 2 and about 100 centipoise.

5. The composition of claim 1, wherein the cellulose based gelling polymer has a viscosity of between about 2 and about 10 centipoise.

6. The composition of claim 1, wherein the composition is dispersible in water within 10 seconds with agitation.

7. The composition of claim 1, wherein the composition disintegrates without the use of water.

8. The composition of claim 1, wherein the active ingredient is susceptible to degradation at temperatures above 50 degrees Celsius.

9. The composition of claim 1, wherein the active ingredient is vitamin C, a botanical product, or a pigment.

10. The composition of claim 1, wherein the composition comprises, by volume, between about 20 and about 99 percent air.

11. The composition of claim 1, wherein the composition comprises, by volume, between about 30 and about 70 percent air.

12. A process of making the composition of claim 1, comprising:
drying a foamed liquid composition comprising the gelling agent at a temperature between about 40 and about 50 degrees Celsius.

13. The process of claim 12, wherein the foamed liquid composition further comprises an active ingredient.

14. The process of claim 12, wherein the foamed liquid composition is dried for between about 10 and about 30 minutes.

15. The process of claim 12, wherein the process does not require the presence of a surfactant.

16. The composition of claim 1, wherein the cellulose based gelling polymer has a viscosity of between about 2 and about 100 centipoise and the composition comprises, by volume, between about 20 and about 99 percent air.

17. the composition of claim 16 wherein the cellulose based gelling polymer is hydroxypropyl methylcellulose, methylcellulose, or a combination thereof
